(19) United States
(12) Patent Application Publication
(10) Pub. No.: US 2006/0134694 A1
(43) Pub. Date: Jun. 22, 2006

(54) Methods of protein profiling by thiolation

(75) Inventor: Mineo Yamakawa, Campbell, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Intel Corporation, Santa Clara, CA (US)

(21) Appl. No.: 11/022,486

(22) Filed: Dec. 22, 2004

(51) Int. Cl. G01N 33/53; G01N 33/00

(52) U.S. Cl. 435/7.1; 530/409; 436/86; 977/904

(57) ABSTRACT

Methods for analyzing a protein are provided, including isolating the protein having original reactive functional groups from a biological sample, and converting substantially all original reactive functional groups into thiol groups, followed by attaching tags to the protein via the thiol groups.

### BACKGROUND OF THE INVENTION

1. Field of the Invention

The invention relates generally to methods useful to detect, analyze and identify the presence of an analyte in a complex biological sample and, more specifically, to methods useful to obtain a protein or peptide profile of such biological sample.

2. Background Information

The ability to accurately profile proteins or peptides is important in many fields, such as protein identification, drug discovery, and many medical diagnostic applications such as early detection of cancers and other critical pathologies. Currently, one typical procedure for protein profiling includes direct detection of protein signature and/or profile without modification or tagging of a protein sample, following only optional preparatory separation and denaturation. Another currently used procedure includes modifying only a single or a few selected functional groups to generate “yes-no” signature or profile (i.e., indicating whether a particular protein was or was not detected). Another technique that is used includes the use of a single or multiple labeled antibodies to identify corresponding specific proteins in a sample.

Raman spectroscopy can be also used for protein profiling. The Raman spectrum of a typical protein molecule comprises about 30 bands occurring in 500-2000 cm⁻¹. These bands are associated with stretching and bending vibrations of the protein main chain or side chains of aromatic amino acids, the C—S and S—S stretching modes of the sulfur-containing amino acids, the carboxyl group modes of the acidic amino acids and various C—C stretching and methyl, methylene, and methyne deformation modes of other amino acids. In addition, a smaller number of broad and overlapping bands in the interval of 2500-3500 cm⁻¹ can be present, due to the hydrogenic stretching modes of protein subgroups, including the environment-sensitive sulfhydryl stretching vibration of the cysteinyl side chain.

### BRIEF DESCRIPTION OF THE DRAWINGS

FIG. 1 is a block-diagram demonstrating the features of the method according to one embodiment of the present invention.

FIG. 2 is a block-diagram demonstrating the features of the method according to another embodiment of the present invention.

FIG. 3 is a block-diagram demonstrating the features of the method according to yet another embodiment of the present invention.

FIG. 4 illustrates a general scheme that can be used to carry out the process of unified thiolization according to one embodiment of the present invention.

FIG. 5 illustrates a reaction scheme that can be used to carry out the process of unified thiolization according to one embodiment of the present invention.

FIG. 6 illustrates a reaction scheme that can be used to carry out the process of thiolization according to one embodiment of the present invention.

FIG. 7 illustrates a reaction scheme that can be used to carry out the process of thiolization according to another embodiment of the present invention.

FIG. 8 illustrates a reaction scheme that can be used to carry out the process of thiolization according to yet another embodiment of the present invention.

FIG. 9 illustrates a general structure of an identifying substance according to one embodiment of the present invention.

FIG. 10 illustrates a general structure of an adduct comprising an identifying substance conjugated to a thiolized protein according to one embodiment of the present invention.

FIG. 11 illustrates a reaction scheme that can be used to carry out the process of conjugating an identifying substance to a thiolized protein according to one embodiment of the present invention.

### DETAILED DESCRIPTION OF THE INVENTION

Terminology and Definitions

For the purposes of the present invention, the following terminology and definitions apply. However, the invention is not limited to merely to the scope of the example definitions given below.

The term “biological sample” or “complex biological sample” may be defined as a sample containing protein-containing analytes, such as a body fluid from a host. The term “body fluid” includes urine, blood, plasma, serum, saliva, semen, stool, sputum, cerebral spinal fluid, tears, mucus, and the like.

The term “protein” may be defined to include proteins, peptides, polypeptides, and molecular compounds with amino acids as well as such protein-containing analytes as antigens, glycoproteins, lipoproteins, and the like. The term “protein” may further include an array of proteins. The term “biomarker” may be defined as a biochemical substance in a body which has a particular molecular feature that makes it useful for measuring the progress of disease or the effects of treatment. Biomarkers can include proteins.

The term “functional group” or “reactive group” may be defined as group of atoms capable, as a whole, to enter chemical reaction(s); in other words, a functional group represents a potential reaction site in an organic compound. The term “original functional group” may be defined as a functional group that is present in a protein, or a denatured protein, prior to the protein or the denatured protein being subjected to the process of thiolization described below.

The term “thiol” may be defined as a sulfur-containing derivative of an alcohol, and having a general formula R—SH, where R is an organic radical, for example, a hydrocarbon-derived radical. The term “thio” or “thio group” may be defined as a sulfur-organic group that is derived from a thiol and having a general formula R—S—. The term “non-thio group” may be defined as a group that is not derived from a thiol.

The term “thiolized” may be defined as a modified protein having more thio groups compared to a protein from which the modified protein is derived. The term “thiolization” may be defined as a process of modification of a protein, the process comprising introducing the thio group(s) in a protein not having such groups prior to modification , or increasing the amount of the thio groups in a protein having some thio groups prior to the modification.

The term “unified thiolization” may be defined as process of thiolization comprising the replacement of at least 98% (mol) of all original and/or modified functional non-thio groups of a protein with the thio groups. The term “partial thiolization” may be defined as process of thiolization comprising the replacement of less than 98% (mol) of all original and/or modified functional non-thio groups of a protein with the thio groups.

The term “thiolizing agent” may be defined as a chemical substance that can be used to react with the original and/or modified non-thio functional groups to convert the latter into the thiol groups. The terms “amino” and “amino group or moiety” may be defined as a group or a moiety having a general formula —N(R)H, where R is hydrogen or an organic radical, for example, a hydrocarbon-derived radical.

The terms “carbonyl” and “carbonyl group or moiety” may be defined as a group or a moiety having a general formula >C═O. The terms “carboxyl” and “carboxyl group or moiety” may be defined as an example of the carbonyl group and refer to a group or a moiety having a general formula —COOH. The terms “carboxyl” and “carboxyl group or moiety” may be further defined to be inclusive of organic acids, and anhydrides, halogen-anhydrides or salts thereof.

The terms “phosphate” and “phosphate group or moiety” may be defined as a group or a moiety having a net formula (PO₄)³⁻, where the atom of phosphorus is bonded with a single bond to each of three atoms of oxygen, and bonded with a double bond to the fourth atom of oxygen. The entire phosphate group has a net negative charge of −3, and may be represented by the general structure O═P(—O—)₃.

The terms “halogen” and “halogen group or moiety” may be defined as a group or a moiety -Hal, where Hal is a halogen atom, i.e., an atom of fluorine, chlorine, bromine or iodine. The terms “imido” and “imido group or moiety” may be defined as a group or a moiety having a general structure —CO—NH—CO— and may be considered either as nitrogen analogues of anhydrides or as diacyl derivatives of ammonia.

The term “aziridine group or moiety” may be defined as a group or a moiety derived from aziridine also known as dimethylenimine. Aziridine is a saturated three-member nitrogen-containing heterocyclic compound.

The terms “acryl or acrylic” and “acrylic group or moiety” may be defined as inclusive of a group or a moiety derived from acrylic acid and a group or a moiety derived from methacrylic acid. Both the group or moiety derived from acrylic acid and from methacrylic acid may be described by a general formula CH₂═CR—CO—, where R is hydrogen (acrylic) or methyl (methacrylic).

The term “adduct” may be defined as a compound formed by an addition reaction between at least two substances. The definition of “adduct” may be inclusive of a compound where the original substances forming the adduct are bound together by any or all kinds of bonds, including ionic bonding, covalent bonding and physical bonding. To illustrate for the proteins discussed in the embodiments of the present invention, one kind of an “adduct” may refer to the product formed by the attachment of thiolating substance, e.g., 2,2′-dithio-bis(ethylamine) to the activated protein so that a new σ-bond is formed between them, and the original conjugation in the activated protein is disrupted.

The term “nanocode” may be defined as a composition that can be used to detect and/or identify a probe physically associated with the composition. In non-limiting examples, a nanocode includes one or more submicrometer metallic barcodes, carbon nanotubes, fullerenes or any other nanoscale moiety that can be detected and identified by scanning probe microscopy. Nanocodes are not limited to single moieties, and in certain embodiments of the invention a nanocode can include, for example, two or more fullerenes attached to each other. Where the moieties are fullerenes, they can, for example, consist of a series of large and small fullerenes attached together in a specific order. The order of differently sized fullerenes in a nanocode can be detected by scanning probe microscopy and used, for example, to identify an attached probe.

The term “composite organic-inorganic particles” or “COINS” may be defined as Raman-active probe constructs that include a core and a surface, wherein the core includes a metallic colloid including a first metal and a Raman-active organic compound. The COINs can further comprise a second metal different from the first metal, wherein the second metal forms a layer overlying the surface of the nanoparticle. The COINs may further comprise an organic layer overlying the metal layer, which organic layer comprises the probe. The term “biotinylated oligonucleotides” may be defined as oligonucleotides modified to include biotinyl groups.

The term “Raman spectroscopy” may be defined as a method of characterization and analysis of the molecule of interest based on the phenomenon of Raman scattering. “Raman scattering” occurs when light passes through a medium of interest and a certain amount of the light gets diverted from its original direction. Some of the scattered light is absorbed by the molecules of the medium resulting in excitation of electrons of the molecules to a higher energy state, followed by light emission at a different wavelength. The difference of the energy of the absorbed light and the energy of the emitted light matches the vibrational energy of the medium. Such difference is a measure of the degree of Raman scattering and can be used for characterization and analysis of the molecule of interest.

The term “surface enhanced Raman spectroscopy” or “SERS” may be defined as a Raman spectroscopy technique having increased sensitivity compared with regular Raman spectroscopy. SERS nanoparticles of some metals to enhance the localized effects of electromagnetic radiation used in Raman spectroscopy. Molecules located in the vicinity of such particles exhibit a much greater sensitivity for Raman spectroscopic analysis. Non-limiting examples of metals nanoparticles of which can be used to perform SERS include gold, silver, or copper.

### Embodiments of the Invention

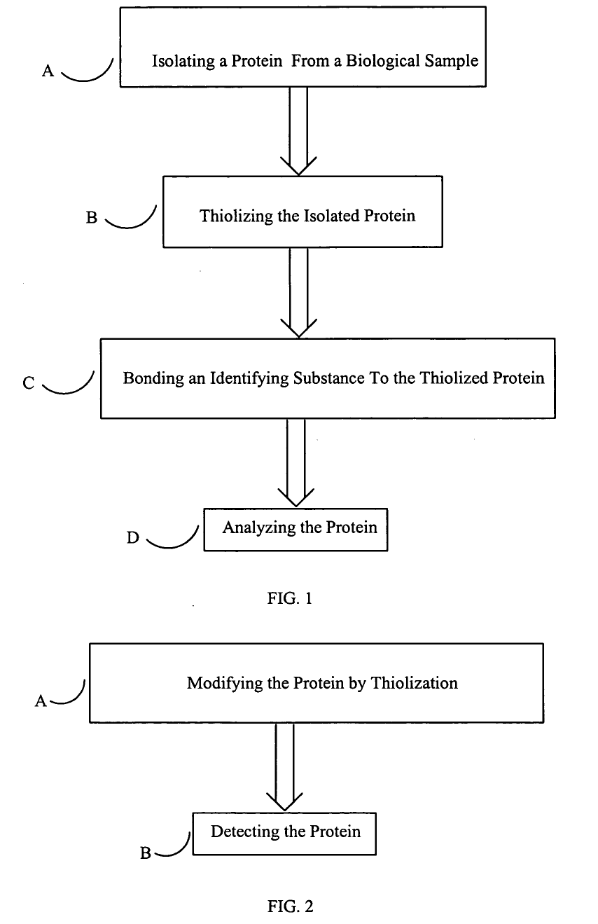

An embodiment of the invention is shown schematically by FIG. 1. According to the embodiment, a method for modifying a protein is provided. The modifying of the protein can be followed by analyzing the protein. The modifying includes optionally isolating the protein having original reactive non-thio functional groups from a biological sample (FIG. 1, A), and subjecting the protein to a process of unified thiolization (FIG. 1, B), or, alternatively, partial thiolization. Analyzing the protein can include bonding an identifying substance to the thiolized protein to form a protein/identifying substance adduct (FIG. 1, C), and subjecting the adduct to an analysis, such as an instrumental analysis (FIG. 1, D). Alternatively, if desired, isolating the protein (FIG. 1, A) mentioned above can be omitted, and the entire blend of proteins can be unifiedly or partially thiolated as long as the quantitative contents of the blend remain unchanged. Any protein and/or peptide and/or any blend or array of proteins and peptides can be analyzed, including biomarkers, antigens (e.g., prostate-specific antigen), interleukins, cardiac troponins, and the like.

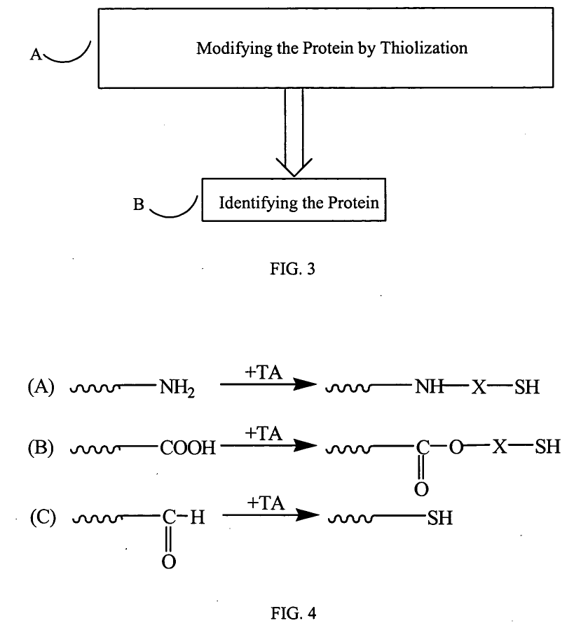

Various approaches can be utilized for analyzing a protein/identifying substance adduct, shown on FIG. 1, D. According to some embodiments, an adduct can be analyzed, to detect the presence of a protein (FIG. 2). According to other embodiments, an adduct can be analyzed, to identify the protein that forms the adduct (FIG. 3). Either the detection (FIG. 2, B) or the identification (FIG. 3, B) or the protein can be accomplished using instrumental methods, as described below.

According to some embodiments of the invention, one method of instrumental analysis that can be used is the method of Raman spectroscopy, where Raman/SERS peak signatures can be used for either detecting or identifying the protein. Examples of some of the moieties and/or molecules providing the peak signatures that can be used include:

(a) intrinsic S—H and SH . . . X moieties of peptides and/or proteins, where X indicates any suitable hydrogen bond acceptor group;

(b) S—H and SH . . . X moieties obtained as a result of protein modifications, including post-translational modifications by thiolation reactions; and

(c) custom designed Raman active molecules tagged to functional group(s) of peptides/proteins through thiolation based conjugation reactions.

The Raman active molecules that can be used include any molecules, which can have inherent strong and unique Raman/SERS peak signatures/profiles and compatible with associated conjugation reactions involving thiolations. For example, acrydite based oligonucleotides and/or fluorescent dyes and/or aromatic amino acids (natural or unnatural) can be conjugated through thiol/sulfur groups of peptides/proteins to generate strong Raman/SERS peak signatures/profiles.

A. Isolation of the Protein (FIG. 1, A).

To isolate the protein from the biological sample, the sample first can be preliminarily treated using standard techniques known to those having ordinary skill in the art. Examples of methods that can be used for the preliminary treatment include high pressure liquid chromatography, capillary electophoresis, ultra-centrifugation, or ultra-filtration, followed by solubilization of the isolated protein in an aqueous buffer solution at conditions such as pH, ionic strength, and temperature to be selected by those having ordinary skill in the art.

Following the preliminary treatment, the protein can be optionally digested, fragmented de-natured. Methods that can be used for the processes of digestion, fragmentation and denaturation are also well known in the art, and include, e.g., enzymatic trypsin digestion, denaturation in solutions having extreme pH or ionic strength, chemical reduction, and the like.

B. Thiolizing the Protein (FIG. 1, B).

To thiolize the isolated protein, a single or a plurality of the protein's original reactive non-thio functional groups can be converted into thiol groups. More than 98% (mol) of the protein's original reactive non-thio functional groups can be converted into thiol groups. Accordingly, the thiolized protein can contain less than about 2% (mol) of the original reactive non-thio functional groups. To accomplish thiolization, the protein can be reacted with at least one thiolizing agent. As a result, the above-mentioned fraction of the original reactive non-thio functional groups of the protein can be converted into the thiol groups.

To conduct the unified or partial thiolization, the original non-thio functional groups of the protein can be chemically converted into the thiol groups. If the process of unified thiolization is used, as a result, art least 98% (mol) of all original non-thio functional groups can be converted into the thiol groups, and a fully thiolized protein can be obtained. A fully thiolized protein can, thus, contain less than about 2% (mol)of the original non-thio reactive functional groups for typical peptides. For diluted small peptides, a fully thiolized protein can contain less than about 0.2% (mol) of the original non-thio reactive functional groups.

Instead of full thiolization, optionally, partial thiolization may be sufficient to enable one having ordinary skill in the art to accomplish further profiling as described above. For example, if more than about 40% of the original non-thio functional groups are thiolized, a profiling could be done. In another example, if efficient and/or strong “signal” tags described below are used, as low as about 10% of the original non-thio functional groups would be needed to be thiolized for tagging in profiling. To convert the original non-thio functional groups into the thiol groups, the protein can be reacted with at least one thiolizing agent.

Typically, the original reactive non-thio functional groups that can be converted into thiol groups include an amino group, a carboxyl group, a carbonyl group, or a phosphate group. Sulfhydryl/thiol groups originally present in the protein can be also utilized at later stages of the process as described below. Step-by-step, sequential thiolization process described below can be used; alternatively, those having ordinary skill in the art can select the process of one-step, simultaneous thiolization, if appropriate.

Examples of the thiolizing agents that can be employed, depending on which original functional group is being thiolized, include succinimidyl acetylthioacetate (SATA), succinimidyl 3-(2-pyridyldithio)propionate (SPDP), 2-iminothiolane (also known as Traut's reagent), 2,2′-dithio-bis(ethylamine)(cystamine), 3-(2-pyridyldithio)propionyl hydrazide (PDPH), succinimidyl trans-4-(maleimidylmethyl)cyclohexane-1-carboxylate, and 2-acetamido-4-mercaptobutyric acid hydrazide. In one embodiment the process of thiolization can include the activation of the protein prior to thiolization. One example of a reagent that can be used for activation is 1-ethyl-3-(3-dimethylaminopropyl)carbodiimide.

To illustrate in general, the process of unified thiolization, can be summarized as shown by FIG. 4. On FIG. 4, the symbol “˜˜˜” stands for the protein less a terminal original and/or modified group(s) of the original functional group, “TA” is the thiolizing agent, and X is a non-thiol portion of the thiolizing agent. FIG. 4 illustrates basic conversion of (A) original and/or modified amino groups (B) original and/or modified carboxyl groups, and (C) original and/or modified group(s) (aldehyde) groups into thiol group(s). The reaction scheme of FIG. 4 illustrates that the totally thiolized protein contains substantially only thiol groups; the original non-thio groups that remain non-converted into the thiol groups are not shown for the illustration purposes.

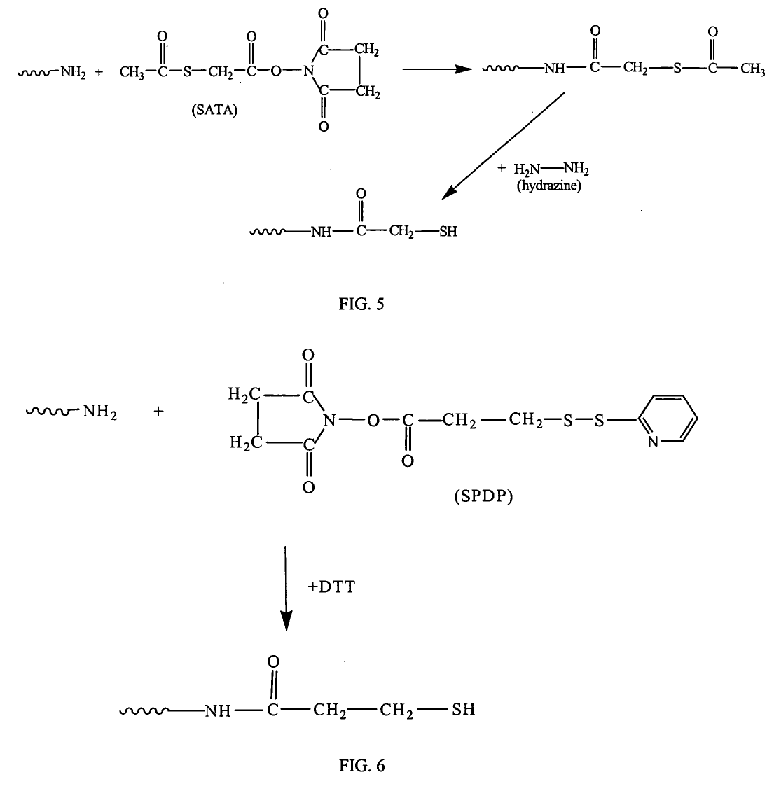

In some embodiments, the process of thiolization can include the final act of de-protection or reduction of a protein adduct. A non-limiting example of a reagent that can be used for de-protection includes hydrazine. The process of unified thiolization which includes using hydrazine as the de-protecting reagent can be illustrated by the reaction scheme shown by FIG. 5. As seen from FIG. 5, the thiolizing agent SATA can be first added to the protein to form an adduct utilizing the original amino groups of the protein. The addition is followed by de-protection to form the totally thiolized protein. One example of a reagent that can be used for de-protection is hydrazine. Those having ordinary skill in the art can select other suitable de-protecting reagents, if desired, and can determine conditions under which the reaction shown by FIG. 5 can be carried out.

Another example of the process of thiolization, also utilizing the original amino groups of the protein, includes addition of the thiolizing agent SPDP, to the protein to form an adduct, followed by reduction using a reducing agent. One example of a reducing agent that can be used is the sulfur-containing reducing agent dithiothreitol (DTT) also known as Cleland's reagent or threo-1,4-dimercapto-2,3-butanediol. DTT has the formula HS—CH₂—CH(OH)—CH(OH—CH₂—SH.

Those having ordinary skill in the art can select other suitable reducing agents, if desired. This thiolization process can be illustrated by FIG. 6. Those having ordinary skill in the art can determine conditions under which the reaction shown by FIG. 6 can be carried out.

In another embodiment, the process of thiolization can include the activation of the protein prior to thiolization. For example, such activation can be used if the original carboxyl or carbonyl functional groups of the protein are utilized. One example of a reagent that can be used for activation is 1-ethyl-3-(3-dimethylaminopropyl) carbodiimide (EDAC). Those having ordinary skill in the art can select other activating agents, if desired.

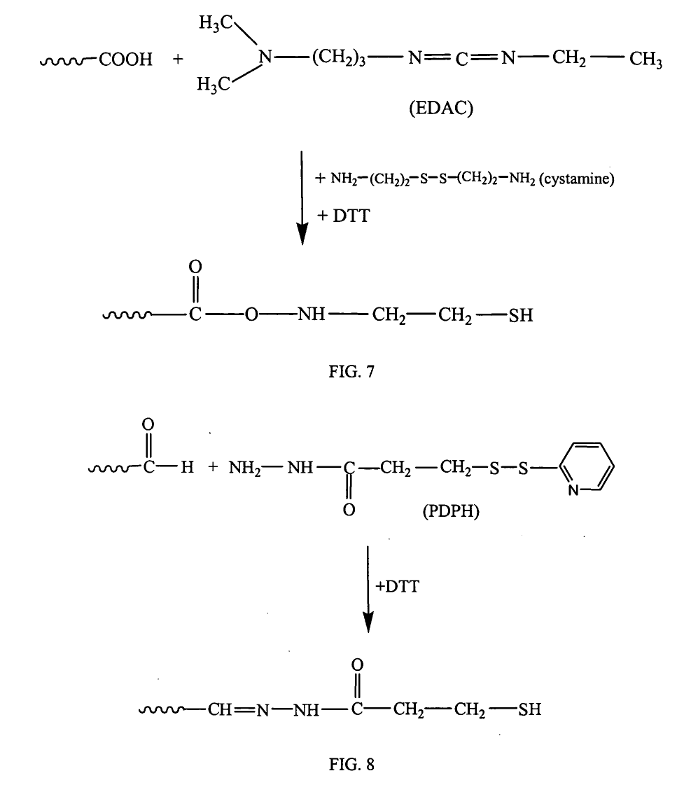

The method can utilize the original carboxyl groups of the protein. To thiolize using one method, the protein can be first activated by adding EDAC to the protein. The activated protein can then be reacted with cystamine, followed by the reduction with a reducing agent such as DTT, to form the totally thiolized protein. Cystamine is a sulfur-containing diamine also known as 2,2′-dithio-bis(ethylamine). In general, this thiolization process can be summarized as illustrated schematically by the reaction shown by FIG. 7. Those having ordinary skill in the art can determine conditions under which the reaction shown by FIG. 7 can be carried out.

Another example of a reagent that can be used for activation is PDPH. As shown by FIG. 8, this method can utilize the original carbonyl (i.e., aldehyde) groups of the protein. To thiolize by the method shown by FIG. 8, the protein can be first activated by adding PDPH which combines the function of both the activating agent and the thiolizing agent. The activation of the protein, therefore, also includes the thiolization. The protein adduct, formed as a result of the reaction between the protein and PDPH, can then be reduced using a reducing agent such as DTT, to form the totally thiolized protein. Those having ordinary skill in the art can determine conditions under which the reaction shown by FIG. 8 can be carried out.

C. Bonding an Identifying Substance to the Thiolized Protein (FIG. 1, C).

According to some embodiments of the present invention, a method for analyzing a can further include bonding at least one identifying substance to the thiolized protein. Such bonding can include chemically conjugating a tagging compound to, or absorbing a tagging compound by, the thiolized protein. The tagging compound can include a reactive moiety and a tag linked to the reactive moiety. The reactive moiety can react with the thiol group of the thiolized protein, thereby bonding the identifying substance to the protein.

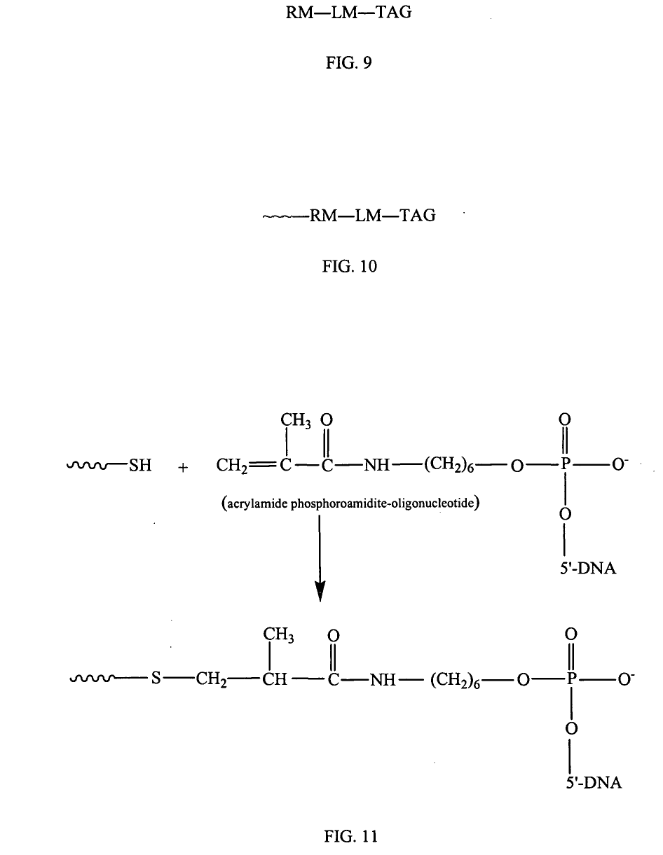

To further illustrate bonding at least one identifying substance to the thiolized protein, the identifying substance can comprise a tag connected to a reactive moiety, as shown schematically by the structure shown by FIG. 9. On FIG. 9, RM symbolizes the reactive moiety of the identifying substance, TAG symbolizes a tag that is used for subsequent protein profiling as discussed below, and LM symbolizes an optional linking moiety connecting the reactive moiety and the tag. In some embodiments, the LM moiety or the RM-LM moiety could be a part of the TAG moiety if LM or RM are detected in profiling, e.g., such as in profiling by Raman/SERS.

In one embodiment, the identifying substance can be bonded to the totally thiolized protein by reacting the reactive moiety of the identifying substance with the thiol groups of the totally thiolized protein. Thus the identifying substance can be chemically conjugated (i.e., by forming a covalent linkage) to the totally thiolized protein, to form a product shown schematically by FIG. 10 One example of a reactive moiety RM (FIG. 10) that can be present in the identifying substance is the acryl group, such as 6-(N-methylacryl)-aminohexyl group. The acryl group, e.g., a substituted acrylamide group can be provided by a product of the ACRYDITE family, available from Matrix Technologies Corp. of Hudson, N.H. Other examples of reactive moieties that can be present in the identifying substance include a halogen, imido group, aziridine group, or acryl group as well as metals, which can react with thiols, e.g., silver, gold, or copper.

In other embodiments, reactive moieties RM can include the moieties derived from compounds containing succinimidyl or maleimido groups, or both succinimidyl and maleimido groups. Specific, non-limiting examples of such compounds include N-succinimidyl(4-iodoacteyl)aminobenzoate, succinimidyl-trans-4-(N-maleimidomethyl)cyclohexane-1-carboxylate) (SMCC), m-maleimidobenzoyl-N-hydroxysuccinimide ester, succinimidyl-4-(p-maleimidophenyl)butyrate, (SMPB) N-γ-(maleimidobutyryloxy) succinimide ester (GMBS), 4-(4-N-maleimidophenyl) butyric acid hydrazide (MPBH), 4-(N-maleimidomethyl)cyclohexane-1-carboxyl-hydrazide (MMCH), succinimidyloxycarbonyl-α-methyl-α-(2-pyridyldithio)toluene, (SMPT), and N-succinimidyl 3-(2-pyridyldithio)propionate (SPDP).

Examples of tags that can be used for conjugating to the protein, via the reactive moiety, include oligonucleotides, such as unmodified, modified or extended oligonuceotides. The modified oligonucleotides that can be used include labels for Raman spectroscopy or surface-enhanced Raman spectroscopy (SERS), and biotinylated oligonucleotides.

To illustrate the conjugating of an identifying substance to a totally thiolized protein, a product of the ACRYDITE family, such as acrylamide phosphoramidite-oligonucleotide can be used. This moiety can react with the thiol groups of the totally thiolized protein as shown by the reaction on FIG. 11.

Acrylamide phosphoramidite-oligonucleotide shown by FIG. 11 includes a reactive moiety (acrylamide), and a tag which is oligonucleotide connected, at its 5′ end, to the reactive moiety, via the hexamethylene phosphate linking moiety. As can be seen from FIG. 11, acryl group of acrylamide phosphoramidite-oligonucleotide can react with the thiol hydrogen of the thiolized protein, leading to bonding the oligonucleotide tag to the protein to be profiled, via the thioether bond that is formed. Those having ordinary skill in the art can determine conditions under which the reaction shown by FIG. 11 can be carried out.

In another embodiment, instead of chemical conjugation, an identifying substance containing the tag can be adsorbed by the protein to be profiled. Some metals such as silver, gold, copper, etc. can form covalent bonds with thiol groups. For example, typically the thiol-gold reaction can be used to immobilize a variety of organic compounds including proteins, peptides, DNA/RNAs, lipids, such those used for fabricating self-assembled monolayers. Also, many proteins and peptides (e.g., albumins) can be directly adsorbed onto metal surfaces through non-specifica binding. Therefore, combination of adsorption and thiol-gold/silver type of system can be used according to some embodiments of the present invention. The tags that can be used include silver colloids, silver nanoparticles, gold colloids, gold nanoparticles, carbon nanotubes, microspheres nanocodes, programmable barcodes, quantum dots (available from Qdot Corporation of Hayward, Calif.), or composite organic-inorganic nanoparticles (COINs). Silver colloids, silver nanoparticles, gold colloids or gold nanoparticles can further include labels for Raman spectroscopy or for SERS.

COINs are readily prepared for use in the invention methods using standard metal colloid chemistry. The preparation of COINs uses the ability of metals to adsorb organic compounds. Indeed, since Raman-active organic compounds are adsorbed onto the metal during formation of the metallic colloids, many Raman-active organic compounds can be incorporated into the COIN without requiring special attachment chemistry.

In general, the COINs used in the invention methods can be prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. This simple type of COIN is referred to as type I COIN. Type I COINs can typically be isolated by membrane filtration. In addition, COINs of different sizes can be enriched by centrifugation.

In alternative embodiments, the COINs can include a second metal different from the first metal, wherein the second metal forms a layer overlying the surface of the nanoparticle. To prepare this type of SERS-active nanoparticle, type I COINs are placed in an aqueous solution containing suitable second metal cations and a reducing agent. The components of the solution are then subject to conditions that reduce the second metallic cations so as to form a metallic layer overlying the surface of the nanoparticle. In certain embodiments, the second metal layer includes metals, such as, for example, silver, gold, platinum, aluminum, and the like. This type of COIN is referred to as type II COINs. Type II COINs can be isolated and or enriched in the same manner as type I COINs. Typically, type I and type II COINs are substantially spherical and range in size from about 20 nm to 60 nm. The size of the nanoparticle is selected to be very small with respect to the wavelength of light used to irradiate the COINs during detection.

Typically, organic compounds, such as oligonucleotides, are attached to a layer of a second metal in type II COINs by covalently attaching the organic compounds to the surface of the metal layer Covalent attachment of an organic layer to the metallic layer can be achieved in a variety ways well known to those skilled in the art, such as for example, through thiol-metal bonds. In alternative embodiments, the organic molecules attached to the metal layer can be crosslinked to form a molecular network.

The COINs used in the invention methods can include cores containing magnetic materials, such as, for example, iron oxides, and the like. Magnetic COINs can be handled without centrifugation using commonly available magnetic particle handling systems. Indeed, magnetism can be used as a mechanism for separating biological targets attached to magnetic COIN particles tagged with particular biological probes.

The metal for achieving a suitable SERS signal is inherent in the COIN, and a wide variety of Raman-active organic compounds can be incorporated into the particle. Indeed, a large number of unique Raman signatures can be created y employing nanoparticles containing Raman-active organic compounds of different structures, mixtures, and ratios. Thus, the methods described herein employing COINs are useful for the simultaneous determination of nucleotide sequence information from more than one, and typically more than 10 target nucleic acids. In addition, since many COINs can be incorporated into a single nanoparticle, the SERS signal from a single COIN particle is strong relative to SERS signals obtained from Raman-active materials that do not contain the nanoparticles described herein. This situation results in increased sensitivity compared to Raman-techniques that do not utilize COINs.

After the identifying substance containing a tag has been bonded to the thiolized protein, the protein can be analyzed (see FIG. 1), for example, using a standard method of instrumental analysis. Examples of instrumental analysis techniques that can be used include Raman spectroscopy, surface-enhanced Raman spectroscopy, mass spectrometry, gel electrophoresis, high pressure liquid chromatography, fluorescence or phosphorescence. As a result of the analysis, the signature or profile of the protein can be obtained.

The following examples are intended to illustrate but not limit the invention.

### EXAMPLE 1

### Preparation and Denaturation of a Sample

A sample can be prepared, separated and delipidated if necessary using any of the methods such as (high performance/pressure liquid chromatography)(HPLC), capillary electrophoresis, ultracentrifugation, or ultrafilteration, followed by solubilization in a buffer solution at appropriate conditions such as pH, temperature, ionic strength, etc., that can be selected by those having ordinary skill in the art. Optionally proteins can be digested, fragmented, and/or denatured, if desired, using such methods as trypsin digestion (enzymatic), denaturation by extreme pH or ionic strength, or chemical reductions.

For example, about 10 μL serum containing about 150 μg of total protein can be mixed with 6M urea and about 5 μL of 200 mM solution of DTT at about 37° C. for about 1 hour. About 20 μL of 200 mM solution of an alkylating reagent can be added and incubated for about 1 hour in the dark, followed by adding about 20 μL of 200 mM solution of DTT at room temperature for about 1 hour. Then, about 800 μL of 25 mM solution of NH₄HCO₃ mixed with 20 μg/50 μL of trypsin can be added and incubates at about 37° C overnight. The reaction can then be stopped by cooling the mixture at about −20° C.

### EXAMPLE 2

### Thiolization Via Carbonyl Group

PDPH (3-(2-pyridyldithiol)propionyl hydrazide) can be used for thiolization of carbonyl groups, e.g., those being present in aldehydes or ketones. In addition, hydrazide-containing agents can be used for thiolization, e.g., AMBH (2-acetamido-4-mercaptobutyric acid hydrazide). The conditions for thiolization process described by a reagent kit supplier can be used. The thiolization reaction can be stopped before proceeding to linking. Following the thiolization reaction, the sample can be lyophilyzed and washed with a buffer solution, such as phosphate buffer at pH of about 7.4.

### EXAMPLE 3

### Thiolization Via Amine Group

Any of SATA (succinimidyl acetylthioacetate), SPDP (succinimidyl 3-(2-pyridyldithio)propionate), or 2-iminothiolane can be used for thiolization of amine groups. The conditions for thiolization process described by a reagent kit supplier can be used Following the thiolization reaction, the sample can be lyophilyzed and washed with a buffer solution, such as phosphate buffer at pH of about 7.4.

### EXAMPLE 4

### Thiolization Via Carboxylic Group

EDAC (1-ethyl-3-(3-dimethylaminopropyl)carbodiimide) can be used for thiolization of carboxylic groups. The conditions for thiolization process described by a reagent kit supplier can be used. Following the thiolization reaction, the sample can be lyophilyzed and washed with a buffer solution, such as phosphate buffer at pH of about 7.4.

### EXAMPLE 5

### Thiolization Via Phosphate Group

Phosphate group can be thiolized via carbodiimide reaction using EDC (1-ethyl-3-(3-dimethyl aminopropyl)carbodiimide hydrochloride) with cystamine. The conditions for thiolization process described by a reagent kit supplier can be used.

### EXAMPLE 6

### Linking

A sample cam be prepared as described in Examples 1-4. Thioether/disulfide bonds such as Acrydite™-based linking can be used for linking TAGs as described by a reagent kit supplier. Alternatively, any other compatible and commercially available linking methods could be used such as through haloacetyl and alkyl halide derivatives (iodoacetyl derivative preferred)—e.g., SIAB (N-succinimidyl(4-iodoacetyl) aminobenzoate); maleimides—e.g., SMCC (succinimidyl-4-(N-maleimidomethyl)cyclohexana-1-carboxylate); MBS (m-Maleimidobenzoyl-N-hydroxysuccimide ester); aziridines; acryloyl and acrylating derivatives; thiol-disulfide exchange reagents, etc. As another alternative, thiol adsorption combined with covalent linking and tagging could be used for attaching TAGs. Any TAGs described above can be used.

Although the invention has been described with reference to the above example, it will be understood that modifications and variations are encompassed within the spirit and scope of the invention. Accordingly, the invention is limited only by the following claims.

What is claimed is:

1. A method for modifying a protein, comprising:
(a) isolating the protein having original reactive non-thio functional groups from a biological sample; and
(b) converting the original reactive non-thio functional groups into thiol groups to obtain a thiolized protein thereby, wherein the thiolized protein contains less than about 2% (mol) of the original reactive non-thio functional groups.

2. The method of claim 1, wherein the thiolized protein is substantially free of the original reactive non-thio functional groups.

3. The method of claim 1, wherein the original reactive functional groups include an amino group, a carboxyl group, a carbonyl group, a sulfhydryl/thiol group, or a phosphate group.

4. The method of claim 1, wherein the converting comprises reacting the protein with at least one thiolizing agent.

5. The method of claim 4, wherein the thiolizing agent is selected from a group consisting of succinimidyl acetylthioacetate, succinimidyl 3-(2-pyridyldithio)propionate, 2-iminothiolane (also known as Traut's reagent), 2,2′-dithio-bis(ethylamine)(cystamine), 3-(2-pyridyldithio)proppionyl hydrazide, succinimidyl trans-4-(maleimidylmethyl)cyclohexane-1-carboxylate, and 2-acetamido-4-mercaptobutyric acid hydrazide.

6. The method of claim 5, wherein the original reactive non-thio functional group is an amino group and the thiolizing agent is selected from a group consisting of succinimidyl acetylthioacetate, succinimidyl 3-(2-pyridyldithio)propionate, and 2-iminothiolane.

7. The method of claim 6, comprising:
(a) reacting the protein with succinimidyl acetylthioacetate to form a protein adduct; and
(b) reacting the adduct with hydrazine.

8. The method of claim 6, comprising:
(a) reacting the protein with succinimidyl 3-(2-pyridyldithio)propionate to form a protein adduct; and
(b) reducing the adduct by reacting the adduct with dithiothreitol.

9. The method of claim 5, wherein the original reactive functional group is a carboxyl group and the thiolizing agent is 2,2′-dithio-bis(ethylamine).

10. The method of claim 9, comprising:
(a) activating the protein by reacting the protein with 1-ethyl-3-(3-dimethylaminopropyl) carbodiimide to form an intermediate;
(b) reacting the intermediate with 2,2′-dithio-bis(ethylamine) to form a protein adduct; and
(c) reducing the adduct by reacting the adduct with dithiothreitol.

11. The method of claim 5, wherein the original reactive non-thio functional group is a carbonyl group and the thiolizing agent is selected from 3-(2-pyridyldithio)proppionyl hydrazide and 2-acetamido-4-mercaptobutyric acid hydrazide.

12. The method of claim 11, comprising:
(a) reacting the protein with 3-(2-pyridyldithio)proppionyl hydrazide to form a protein adduct; and
(b) reducing the adduct by reacting the adduct with dithiothreitol.

13. The method of claim 5, wherein the original reactive non-thio functional group is a phosphate group and the thiolizing agent is 2,2′-dithio-bis(ethylamine).

14. The method of claim 13, further comprising:
(a) activating the protein by reacting the protein with 1-ethyl-3-(3-dimethylaminopropyl)carbodiimide to form an intermediate;
(b) reacting the intermediate with 2,2′-dithio-bis(ethylamine) to form a protein adduct; and
(c) reducing the adduct by reacting the adduct with dithiothreitol.

15. A method for detecting a protein, comprising modifying a protein according to claim 1 to obtain a thiolized protein, and further comprising bonding at least one identifying substance to the thiolized protein to obtain a protein/identifying substance adduct, and subjecting the adduct to an analysis, to detect the presence of the protein.

16. The method of claim 15, wherein the bonding the identifying substance comprises chemically conjugating a tagging compound to the thiolized protein.

17. The method of claim 16, wherein the tagging compound comprises a reactive moiety and a tag linked to the reactive moiety, wherein the reactive moiety reacts with the thiol group of the thiolized protein, thereby bonding the identifying substance to the protein.

18. The method of claim 17, wherein the reactive moiety includes a halogen, imido group, aziridine group, or acryl group a halogen, imido group, aziridine group, acryl, silver, gold, or copper.

19. The method of claim 17, wherein the reactive moiety is 6-(N-methylacryl)-aminohexyl.

20. The method of claim 17, wherein the reactive moiety is derived from a product selected from a group consisting of N-succinimidyl(4-iodoacteyl)aminobenzoate, succinimidyl-4-(N-maleimidomethyl)cyclohexane-1-carboxylate), m-maleimidobenzoyl-N-hydroxysuccinimide ester, succinimidyl-4-(p-maleimidophenyl)butyrate, (SMPB) N-γ-(maleimidobutyryloxy)succinimide ester (GMBS), 4-(4-N-maleimidophenyl) butyric acid hydrazide (MPBH), 4-(N-maleimidomethyl)cyclohexane-1-carboxyl-hydrazide (MMCH), succinimidyloxycarbonyl-α-methyl-α-(2-pyridyldithio)toluene, (SMPT), and N-succinimidyl 3-(2-pyridyldithio)propionate (SPDP).

21. The method of claim 17, wherein the tag comprises oligonucleotides, silver colloids, silver nanoparticles, gold colloids, gold nanoparticles, carbon nanotubes, microspheres nanocodes, programmable barcodes, quantum dots, or composite organic-inorganic nanoparticles.

22. The method of claim 21, wherein the oligonucleotides comprise modified or extended oligonuceotides.

23. The method of claim 22, wherein the modified oligonucleotides include labels for Raman spectroscopy or surface-enhanced Raman spectroscopy.

24. The method of claim 22, wherein the modified oligonucleotides include biotinylated oligonucleotides.

25. The method of claim 21, wherein any of the silver colloids, silver nanoparticles, gold colloids or gold nanoparticles further include labels for Raman spectroscopy or surface-enhanced Raman spectroscopy.

26. The method of claim 15, wherein the bonding the identifying substance comprises adsorbing a tagging compound by the thiolized protein.

27. The method of claim 1, wherein the proteins include biomarkers, antigens, interleukins, or cardiac troponins.

28. The method of claim 15, wherein the analysis is an instrumental analysis.

29. The method of claim 28, wherein the instrumental analysis includes Raman spectroscopy, surface-enhanced Raman spectroscopy, mass spectrometry, gel electrophoresis, high pressure liquid chromatography, fluorescence or phosphorescence.

30. The method of claim 15, wherein the protein comprises an array of proteins.

31. A method for identifying a protein, comprising modifying a protein according to claim 1 to obtain a thiolized protein, and further comprising bonding at least one identifying substance to the thiolized protein to obtain a protein/identifying substance adduct, and subjecting the adduct to an analysis, to identify the protein.

32. The method of claim 31, wherein the bonding the identifying substance comprises chemically conjugating a tagging compound to the thiolized protein.

33. The method of claim 32, wherein the tagging compound comprises a reactive moiety and a tag linked to the reactive moiety, wherein the reactive moiety reacts with the thiol group of the thiolized protein, thereby bonding the identifying substance to the protein.

34. The method of claim 33, wherein the reactive moiety includes a halogen, imido group, aziridine group, or acryl group a halogen, imido group, aziridine group, acryl, silver, gold, or copper.

35. The method of claim 33, wherein the reactive moiety is 6-(N-methylacryl)-aminohexyl.

36. The method of claim 33, wherein the reactive moiety is derived from a product selected from a group consisting of N-succinimidyl(4-iodoacteyl)aminobenzoate, succinimidyl-4-(N-maleimidomethyl)cyclohexane-1-carboxylate), m-maleimidobenzoyl-N-hydroxysuccinimide ester, succinimidyl-4-(p-maleimidophenyl)butyrate,(maleimidobutyryloxy)succinimide (SMPB) N-γ-ester (GMBS), 4-(4-N-maleimidophenyl)butyric acid hydrazide (MPBH), 4-(N-maleimidomethyl)cyclohexane-1-carboxyl-hydrazide (MMCH), succinimidyloxycarbonyl-α-methyl-α-(2-pyridyldithio)toluene, (SMPT), and N-succinimidyl 3-(2-pyridyldithio)propionate (SPDP).

37. The method of claim 33, wherein the tag comprises oligonucleotides, silver colloids, silver nanoparticles, gold colloids, gold nanoparticles, carbon nanotubes, microspheres nanocodes, programmable barcodes, quantum dots, or composite organic-inorganic nanoparticles.

38. The method of claim 37, wherein the oligonucleotides comprise modified or extended oligonuceotides.

39. The method of claim 38, wherein the modified oligonucleotides include labels for Raman spectroscopy or surface-enhanced Raman spectroscopy.

40. The method of claim 38, wherein the modified oligonucleotides include biotinylated oligonucleotides.

41. The method of claim 37, wherein any of the silver colloids, silver nanoparticles, gold colloids or gold nanoparticles further include labels for Raman spectroscopy or surface-enhanced Raman spectroscopy.

42. The method of claim 31, wherein the bonding the identifying substance comprises adsorbing a tagging compound by the thiolized protein.

43. The method of claim 31, wherein the proteins include biomarkers, antigens, interleukins, or cardiac troponins.

44. The method of claim 31, wherein the analysis is an instrumental analysis.

45. The method of claim 44, wherein the instrumental analysis includes Raman spectroscopy, surface-enhanced Raman spectroscopy, mass spectrometry, gel electrophoresis, high pressure liquid chromatography, fluorescence or phosphorescence.

46. The method of claim 31, wherein the protein comprises an array of proteins.

47. A composition, comprising an identifying moiety bonded to a thiolized protein, wherein the thiolized protein contains less than about 2% of reactive non-thio functional groups.

48. The composition of claim 47, wherein the thiolized protein is substantially free of the reactive non-thio functional groups.

49. The composition of claim 47, wherein the identifying moiety is chemically conjugated to the thiolized protein.

50. The composition of claim 49, wherein the identifying moiety is conjugated via the thiol group of the thiolized protein.

51. The composition of claim 49, wherein the identifying moiety is derived from a substance comprising a reactive group and a tag linked to the reactive group.

52. The composition of claim 51, wherein the reactive group includes a halogen, imido group, aziridine group, or acryl group.

53. The composition of claim 51, wherein the reactive group is 6-(N-methylacryl)-aminohexyl.

54. The composition of claim 51, wherein the reactive group is provided by a product selected from N-succinimidyl(4-iodoacteyl)aminobenzoate, succinimidyl-4-(N-maleimidomethyl)cyclohexane-1-carboxylate), m-maleimidobenzoyl-N-hydroxysuccinimide ester, succinimidyl-4-(p-maleimidophenyl)butyrate, (SMPB) N-γ-(maleimidobutyryloxy)succinimide ester (GMBS), 4-(4-N-maleimidophenyl)butyric acid hydrazide (MPBH), 4-(N-maleimidomethyl)cyclohexane-1-carboxyl-hydrazide (MMCH), succinimidyloxycarbonyl-α-methyl-α-(2-pyridyldithio)toluene, (SMPT), and N-succinimidyl 3-(2-pyridyldithio)propionate (SPDP).

55. The composition of claim 51, wherein the tag comprises oligonucleotides, silver colloids, silver nanoparticles, gold colloids, gold nanoparticles, carbon nanotubes, microspheres nanocodes, programmable barcodes, quantum dots, or composite organic-inorganic nanoparticles.

56. The composition of claim 55, wherein the oligonucleotides comprise modified or extended oligonuceotides.

57. The composition of claim 56, wherein the modified oligonucleotides include labels for Raman spectroscopy or surface-enhanced Raman spectroscopy.

58. The composition of claim 56, wherein the modified oligonucleotides include biotinylated oligonucleotides.

59. The composition of claim 55, wherein any of the silver colloids, silver nanoparticles, gold colloids or gold nanoparticles further include labels for Raman spectroscopy or surface-enhanced Raman spectroscopy.

60. The composition of claim 30, wherein the identifying moiety is adsorbed by the thiolized protein.

61. The composition of claim 47, wherein the proteins include biomarkers, antigens, interleukins, or cardiac troponins.

62. A composition, comprising a thiolized protein, wherein the thiolized protein contains less than about 2% of non-thio reactive functional groups.

63. The composition of claim 62, wherein, the thiolized protein is substantially free of the reactive non-thio functional group.
